(19) United States
(12) Patent Application Publication
(10) Pub. No.: US 2018/0218737 A1
(43) Pub. Date: Aug. 2, 2018

(54) SYSTEM AND METHOD FOR PERFORMING REAL TIME ANALYSIS OF DICTATED REPORTS FOR IMPROVING COMPLIANCE EFFICIENCY

(71) Applicant: University of Maryland, Baltimore, Baltimore, MD (US)

(72) Inventor: Edward HERSKOVITS, Baltimore, MD (US)

(73) Assignee: University of Maryland, Baltimore, Baltimore, MD (US)

(21) Appl. No.: 15/883,642

(22) Filed: Jan. 30, 2018

Related U.S. Application Data
(60) Provisional application No. 62/451,854, filed on Jan. 30, 2017.

(51) Int. Cl. G10L 15/26; G06F 17/24; G11B 27/028; G16H 10/60

(52) U.S. Cl. CPC G10L 15/265; G06F 17/243; G16H 10/60; G06F 17/248; G11B 27/028

(57) ABSTRACT

A method for real-time validation of a dictated report dictated by a user including receiving, for each of a plurality of report types, report-type criteria that define the report type and compliance rules associated with the report type, receiving report text from a composition system in which the voice of the user is transcribed as the report text in real-time as the user dictates and/or types the dictated report, extracting metadata from the received report text, determining a report type of the dictated report by comparing the extracted metadata to the report-type criteria received for each of the plurality of report types, determining compliance with the compliance rules associated with the determined report type by applying the compliance rules to the received report text, and transmitting, to a user-interface, information indicating the determined compliance with the compliance rules associated with the determined report type, and making additional best-practice recommendations if relevant findings are described in the report text.

The present application claims priority to the earlier filed provisional application having Ser. No. 62/451,854, and hereby incorporates the subject matter of the provisional application in its entirety.

### BACKGROUND OF THE INVENTION

### 1. Field of the Invention

The present invention relates to a system and method for performing real time analysis of dictated reports. The field of Radiology is used for the purposes of illustration of this method.

### 2. Description of Related Art

Radiologists often dictate the results of their findings to create digital reports for the studied image or set of images. FIG. 1 shows a typical work situation where the radiologist sits in front of three computer monitors displaying a patient's images, data associated with the images, descriptive text, etc. Although only three monitors are shown in FIG. 1, some radiologists use a different number of monitors according to needs and preferences. The view from the radiologist's perspective is shown in FIG. 2. The image may be acquired by any of the many medical imaging procedures available, such as, X-ray transmission, CT, MRI, PET, ultrasound, etc.

A radiologist creates one or more text-based digital reports regarding the observed images and medically relevant findings regarding the patient. While the digital report may be created via a keyboard, for most radiologists dictating the reports is significantly more convenient than editing via the keyboard.

The radiologist may orally describe the results of a radiology study using a dictation device that records the oral description in a speech or sound digital file. The sound digital file may be stored on a digital drive such as a memory, a hard drive, a DVD or a CD. The sound digital files may be then transcribed to one or more text files by “speech recognition software” such as Dragon®, as commercialized by the Nuance Communications, Inc. Thus, the reports dictated by the radiologist, with respect to the imaging procedures performed and the findings/diagnostic corresponding to a patient, are transcribed into text, thereby forming digital text reports corresponding to the imaging procedures and findings corresponding to the patient.

Such digital text reports may be later consulted by the plurality of parties contributing to providing healthcare to the patient, such as: physicians directly providing health services to the patient, supervisors of the physicians, researchers, insurance companies, for example Medicare, parties involved in litigation, attorneys, etc.

One of the problems encountered by radiologists is that they have to ensure that the dictated radiology reports include the following: all the specific pieces of information required by various laws and regulations relating to healthcare services (e.g., FDA regulations); all the specific pieces of information required by insurance companies and Medicare; and all the specific pieces of information required by the rules and regulations of the medical provider (e.g., hospital, radiology department etc.). These non-clinical requirements may distract the radiologist from the primary task of producing clinically accurate and timely reports.

Further, the radiologist may want to include all the information necessary to the plurality of parties that may want and need to consult the reports in the future (e.g., physicians directly providing health services to the patient, supervisors of the physicians, researchers, insurance companies, parties involved in litigation, attorneys, etc.).

For example, a radiology report regarding a mammogram in which the radiologist observes a cyst must include the dimensions of the cyst; a radiology report generated by a trainee (e.g., a resident or fellow) must include a statement that the attending physician agrees with the trainee's report.

Additionally, some radiology reports must include a half-dozen or more such individual pieces of information. For example, an ultrasound study of a patient who is in the first trimester of pregnancy must include an adequate history, in addition to descriptions of the amniotic fluid, the placenta, the ultrasound technique, the Doppler technique, the ultrasound and Doppler findings, and a teaching statement.

There are several negative consequences of creating incomplete reports. For instance, if a report created by a radiologist does not comply with the various requirements (e.g., does not list all the necessary pieces of information), the report may not be accepted by the insurance company. As a result, payments will be delayed or potentially never recovered, the report will be returned to the radiologist (usually after review by a practice administrator to determine the exact deficiencies in the report), which leads to waste of time and ultimately to increase of the cost of healthcare. Additionally, extensive non-compliance may lead to penalties. Thus, when dictating a radiology report, the radiologist is under continuous pressure to ensure that all the specific pieces of information needed for compliance are included in the report.

Conventionally, most practices employ at least one full-time employee at significant cost in order to review reports before submission to an insurance company. However, if a deficiency in a report is discovered in this manner, it is typically discovered days or weeks after the original dictation before being returned to the radiologist. This is not only inefficient but increases the burden on the radiologist to revisit and edit dictations well after the original dictation date, thereby increasing work load on the radiologist.

Accordingly, there is a demand for real-time compliance analysis of dictated reports without the need for further manual review before submission to an insurance company.

The invention disclosed herein comes as a solution to the above problems regarding the dictation of radiology reports and as a tool helping radiologists and other professionals to more easily and safely create reports compliant with various laws, rules and regulations.

The above information disclosed in this overview section is only for enhancement of understanding of the background of the invention and therefore it may contain information that does not form any part of the prior art.

### BRIEF SUMMARY OF THE INVENTION

An embodiment of the present invention relates to a method for real-time validation of a dictated report dictated by a user. The method including receiving, for each of a plurality of report types, report-type criteria that define the report type and compliance rules associated with the report type, receiving report text from a composition system in which the voice of the user is transcribed as the report text in real-time as the user dictates the dictated report, extracting metadata from the received report text, determining a report type of the dictated report by comparing the extracted metadata to the report-type criteria received for each of the plurality of report types, determining compliance with the compliance rules associated with the determined report type by applying the compliance rules to the received report text, and transmitting, to a user-interface, information indicating the determined compliance with the compliance rules associated with the determined report type.

Another embodiment of the present invention related to a system for real-time validation a dictated report dictated by a user. The system including a composition system in which a voice of the user is transcribed as report text in real-time as the user dictates the dictated report, and a processor programmed to perform steps of receiving, for each of a plurality of report types, (i) report-type criteria that define the report type and (ii) compliance rules associated with the report type, receiving report text from a composition system in which the voice of the user is transcribed as the report text in real-time as the user dictates the dictated report, extracting metadata from the received report text, determining a report type of the dictated report by comparing the extracted metadata to the report-type criteria received for each of the plurality of report types, determining compliance with the compliance rules associated with the determined report type by applying the compliance rules to the received report text, and transmitting, to a user-interface, information indicating the determined compliance with the compliance rules associated with the determined report type.

### BRIEF DESCRIPTION OF THE DRAWINGS

FIG. 1 shows a typical work situation where the radiologist sits in front of three computer monitors displaying a patient's images.

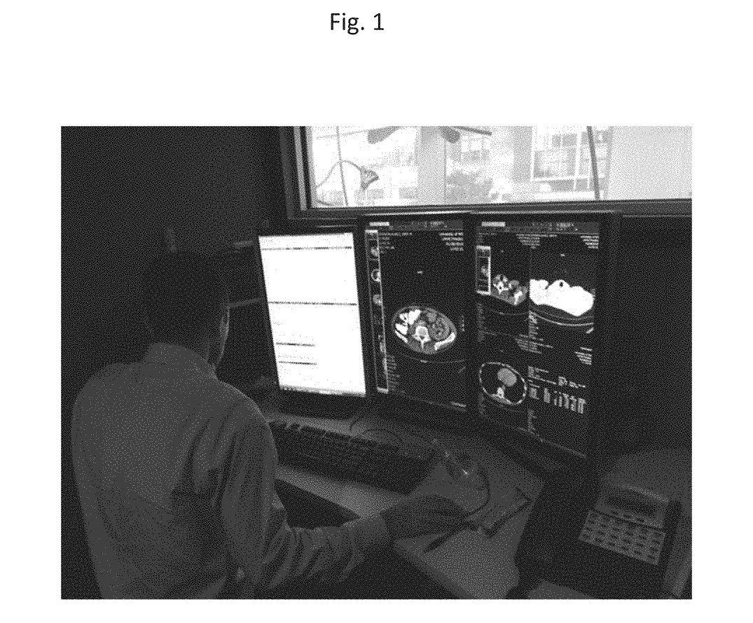

FIG. 2 shows a view from the radiologist's perspective of the typical work situation shown in FIG. 1.

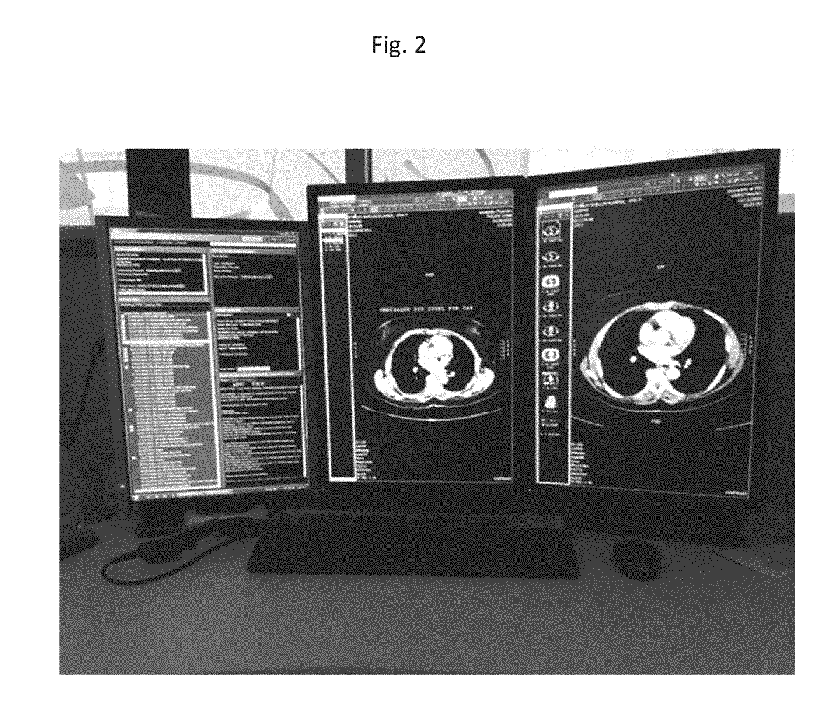

FIG. 3 shows a system according to an exemplary embodiment of the invention.

FIG. 4 shows a monitor displaying a patient's image and another monitor displaying transcribed dictated text.

FIG. 5 shows how the software may cause a compliance window to display a certain background color according to the result of the compliance analysis.

FIG. 6 shows a schematic diagram of the basic operations of Aulev™ software.

FIG. 7 shows another schematic diagram of the basic operations of Aulev™ software.

### DETAILED DESCRIPTION OF THE INVENTION

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

Hereinafter, a system and method for performing real time analysis of dictated reports to improve compliance with insurance and professional standards and to improve efficiency. This invention may, however, be embodied in many different forms and should not be construed as limited to the exemplary embodiments set forth herein. Rather, these exemplary embodiments are provided so that this disclosure is thorough, and will fully convey the scope of the invention to those skilled in the art.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Further, it will be understood that when an element is referred to as being “connected to” another element, it can be directly connected to the other element, or intervening elements may be present. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

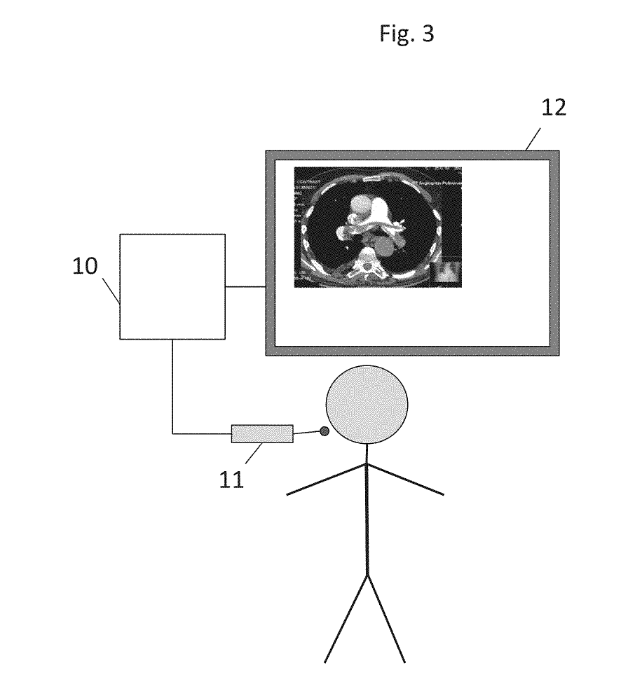

According to an exemplary embodiment of the invention shown in FIG. 3, a system for performing real time analysis of dictated report may include: a voice recorder 11; a computer system 10; and one or more computer monitors 12.

The voice recorder 11 may be configured to record reports dictated by a physician and to create, in real-time, digital audio files (e.g., “wav” or “mp3” files) corresponding to the dictated report. The report may be dictated by a radiologist and may include information with regard to the images acquired by various medical imaging procedures performed on a patient. The images may be displayed on one or more computer displays. The reports dictated by the radiologist may regard the images displayed on the monitor (and observed by the radiologist), the performed imaging procedures, the observations/findings/diagnostic corresponding to the images and the patient, etc.

The computer system 10 may be connected to the voice recorder 11 (e.g. via wired connection or wirelessly such as via Bluetooth® connection) and may be configured to receive, in real-time, from the voice recorder the digital audio signals corresponding to the dictated reports.

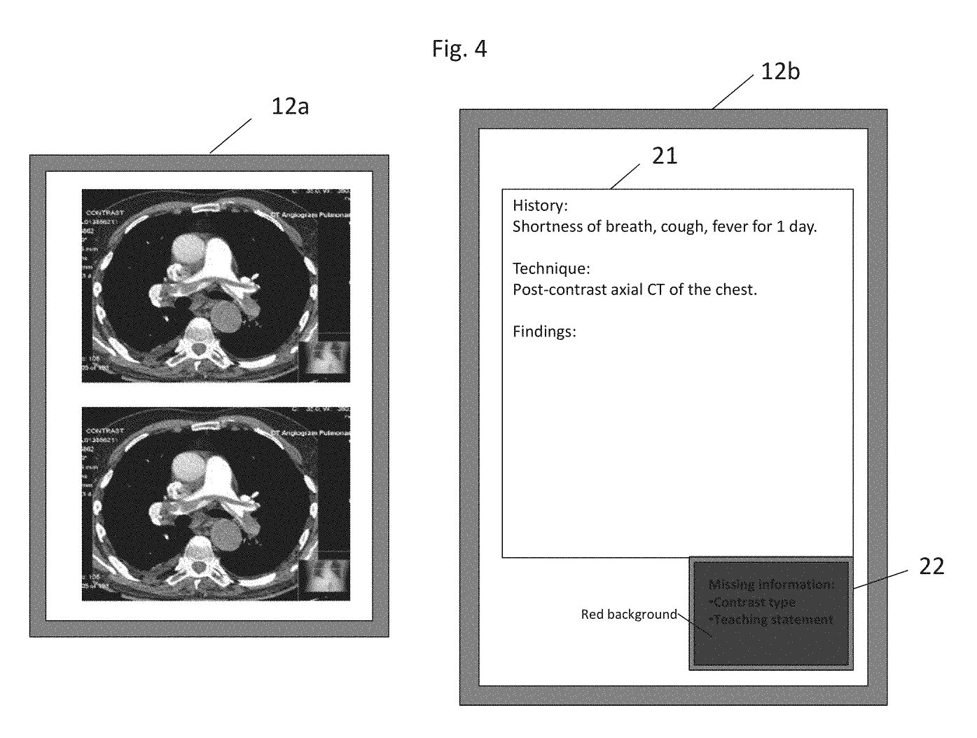

The computer system may include one or more software modules configured to perform transcription in real-time of the audio digital files (including the oral reports dictated by radiologist or other physician) to text files. The software modules may include “speech recognition software” (e.g., the software “Dragon®” commercialized by the Nuance Communications, Inc. the PowerScribe® 360 software). Thus, reports dictated by the radiologists (with respect to the images observed by the radiologist, the performed imaging procedures, the observations/findings/diagnoses corresponding to the images and the patient) may be transcribed into written text, thereby forming digital text reports. The computer system may be configured to display the transcribed dictated text on the monitor 12b in real time as shown by the report text 21 shown in FIG. 4.

The software modules may further include a module configured to determine whether a digital text report is complete and complies with the specific rules and requirements that may apply (hereinafter referred as “compliance-determination-module”). The inventor herein has implemented such a software module for real-time analysis of various types of radiology reports (the software is named Aulev™). Operations and functions of Aulev™ will be described later.

The compliance-determination-module is configured to determine the type of report to be analyzed and processed. Examples of report types are: reports for mammograms; reports for PET scans of the brain; reports for catheter angiography of the lower extremity or heart; reports for MRI of the spine, reports for sonography of a woman in the first trimester of pregnancy.

The compliance-determination-module may determine the type of report to be analyzed and processed according to an entry received from the physician. Alternatively, the compliance-determination-module may receive, as input, report-type criteria defining numerical, text, or combined criteria that define a report type, and the compliance-determination-module may determine the type of report to be analyzed by comparing the inputted report-type criteria to the digital text report.

For example, the report-type criteria could be organized as a look-up table or a series of rules, allowing the compliance-determination-module to quickly and efficiently determine a report type from data extracted from the digital text report. However, it should understand that report-type criteria may be organized according to other well-known techniques.

The compliance-determination module is able to perform real-time analysis of the digital text file and extract data which then may be compared to the report-type criteria in order to determine the type of report to be analyzed and processed.

The report-type criteria may be included in a “report-type database” in the computer or externally in a server or the like along with corresponding information that such reports need to contain in order to achieve compliance, that is, compliance rules corresponding to the report type. The compliance-determination module may obtain the compliance rules corresponding to the report type at the same time or at a different time as receiving the report-type criteria.

For each report type, the compliance rules corresponding to the report type may be organized, for example, as report-validation requirements defining numerical, text, or combined criteria that associate validation requirements for the report type and report-validation criteria defining natural language processing, numerical, text, or combined criteria for each of the requirements included in the report-validation requirements for the report type.

For instance, the report type database may include a first type of report (e.g., mammogram report) and the corresponding compliance rules for the first type of report, the database may include a second type of report (e.g., brain PET scan report) and the corresponding compliance rules for the second type of report, etc. The report-type database may be edited to add more report-types or to change the compliance rules (e.g., add new rules, delete rules, modify rules) corresponding to a certain report-type according to circumstances. In addition, the database contains teaching compliance rules for all reports generated during the course of teaching a medical trainee (e.g., resident or fellow).

Additionally, the compliance-rules may include, for each of the report types, recommendation-applicability criteria defining numerical, text, or combined criteria that determine recommendations applicable to the report-type and data extracted from the digital text file and recommendation-recommendation criteria defining natural language processing, numerical, text, or combined criteria for each of the recommendations applicable to the report-type.

Further, the compliance-rules may be defined in a machine-readable format so as to be compatible with the compliance-determination module without extensive processing by the compliance-determination module.

The compliance-rules may be defined by a human or defined by a processor programmed to determine associations between report text, extracted data from report text, and report validity, such as a machine-learning or statistical application.

The system according to an embodiment of the invention may also incorporate recommendation rules, which encode best practices for recommended follow-up, or additional testing for certain findings described in a report.

The system according to an embodiment of the invention may be configured to allow the operator or another person to edit the criteria contained within the compliance rules, as necessary.

The compliance-determination-module is configured to perform text analysis of the text in a digital text file or in a text-editing application in order to determine compliance of the text with a set of compliance-rules selected from the database.

For example, after determining the type of report to be analyzed and processed using the report-type criteria obtained from the report-type database, the compliance-determination module may obtain report-validation requirements and report-validation criteria corresponding to the determined type of report. Then, the compliance-determination module may apply the report-validation criteria corresponding to each of the report-validation requirements for the determined type of report in real-time to the digital text file or data extracted from the digital text file or text-editing window in order to determine compliance with the report-validation requirements. After determining compliance with each of the report-validation requirements, the compliance-determination module may transmit a list of satisfied and non-satisfied criteria to user-interface.

For example, as shown with reference to FIG. 4, the compliance-determination module is configured to display on a computer display 12b (may be the same as the computer display on which the medical images and/or report are displayed) a compliance-window 22 and to change the color and the content displayed on the compliance-window according to the result of the text analysis.

The compliance-window may display a red background if the text analysis indicates that the dictated text does not comply with all relevant rules, and may display a green background if the text analysis indicates that the dictated text does comply with the rules. Further, the compliance-window may display text informing the radiologist what type of information is missing and needs to be provided in order to complete the report, e.g., the compliance window may show a listing of non-satisfied criteria. The software changes this display in real-time as the radiologist dictates, ensuring that any deficiencies in the dictated report are detected as early as possible.

Additionally, when receiving recommendation-applicability criteria and recommendation-recommendation criteria (e.g., follow-up criteria) after determining the type of report to be analyzed and processed using the received report-type criteria, the compliance-determination module may apply the recommendation-recommendation criteria corresponding to each of the recommendations applicable to the determined type of report according to the recommendation-applicability criteria to the digital text file, text-editing window, or data extracted from the digital text file in order to determine recommendations to be included in the report being dictated. The determined recommendations may then be transmitted by the compliance-determination module to user-interface.

The functioning of the above described system for performing real-time analysis of dictated report is described with respect to the way a radiologist would use the system in the following.

Upon receiving and studying a radiologic image or set of images (e.g., images displayed on a computer display) the radiologist starts creating a dictated report by activating/pushing one or more controls on a computer interface and/or by speaking into the voice recorder, or by typing directly into the report. The radiologist may dictate one report for more than one examination; for example, a CT of the entire spine may consist of 3 examinations: cervical spine, thoracic spine, and lumbar spine. The radiologist may choose to dictate one comprehensive report for all three examinations at the same time, which may increase the complexity of reporting requirements. The software may detect the type of report from the transcription software, from the image display software, the radiology information system, or the hospital information system (for example, software Aulev™ currently implemented by inventor does not require the radiologist to enter anything about the report; instead Aulev™ contacts PowerScribe® to obtain all this information when the radiologist begins a new report). The voice recorder may record the string of words/sounds spoken by the radiologist and may transmit in real-time the string of words to the computer as digital voice files. The computer may receive from the voice recorder the string of words (as digital voice files) and may transcribe in real-time (as the words are spoken by the radiologist) the string of words into digital text files or a text-editing window. The string of words (i.e. the partial report dictated up to that moment) may be displayed, in real-time, in a window on the computer display (e.g., see text displayed in window 21 of display 12b).

Further, the compliance-determination-software may select from the database the compliance rules corresponding to the report type(s) obtained from the PACS, RIS, or from the dictation software. Then, the compliance-determination-software may analyze the string of words and determine whether the string of words (i.e., the report dictated up to that moment) complies with the selected compliance rules. For instance, the compliance-determination-software may determine whether the report includes a certain piece of information specified by one of the compliance rules, the software may determine whether a certain number (e.g., age) is in the range specified by the compliance rules, etc.

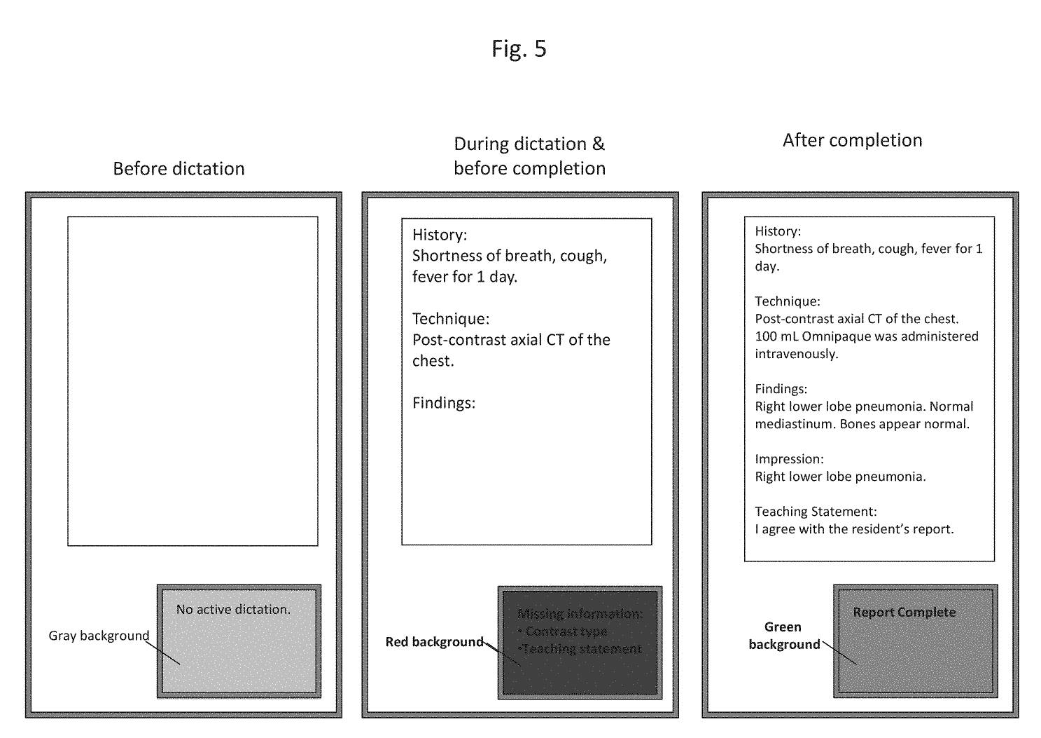

The software may cause the compliance window to display a certain background color according to the result of the compliance analysis. For example, as shown with reference to FIG. 5, the background color may be gray prior to the start of dictation, indicating inactivity: the background color may be red during dictation if the dictated text does not comply with the compliance rules (i.e. the report is incomplete); the background color may be changed from red to green when the dictated text complies with all rules (i.e., the report is complete); and the background color may be changed to blue to indicate one or more best-practice recommendations for follow-up or additional tests, including that no additional test is required for a particular finding.

Further, the software may cause the compliance window to display text (e.g., over the red background) indicating the type of missing information, such that the radiologist can easily view the type of information he needs to dictate in order for the report to be complete.

The entire process described above may be performed in real time and may take a very short time (e.g., approximately 200 ms or less). As the radiologist dictates the report, the computer may periodically update (e.g., every second, or every 0.5 seconds, in real-time) the text displayed on the monitor as the text continues to be dictated by the radiologist, or as the radiologist edits the report using the keyboard. Further, as the radiologist dictates the report and the dictated text is transcribed into digital text, the status of the compliance window (e.g., the color of the background, the text displayed by the compliance-window) may be periodically updated (e.g., every second, or every 0.5 seconds, in real-time). Thus, the radiologist can see in real-time the report text he dictated up to that moment and the compliance-window including the color of the background and the information needed, if any, to achieve compliance. By observing the compliance window the radiologist knows whether the dictated report is complete or incomplete (e.g., if the report is complete or incomplete the background is green or red, respectively).

Further, if the report is incomplete the radiologist can see the type of information needed for compliance (i.e., information displayed on the compliance-window) and can supply the missing information by dictation or keyboard.

The software is further configured to output a report, such as a pdf or text file, for use in filing and recordkeeping.

The inventor herein has implemented a system, method and application as described above for various types of radiology reports. The application is called Aulev™ and works as described in the following.

Aulev™ is launched when a user logs in to a workstation; it's always running when the Radiologist dictates a case. The default location of the main Aulev™ window is the bottom of the primary monitor, which works well for most users; the user can move the Aulev™ window to any location that's convenient.

Aulev™ continuously runs a loop, with the following steps:
1. Establish a connection with the dictation software.
2. Obtain a list of currently dictated examinations from the dictation software.
3. If a connection could not be established (i.e., the Radiologist is not currently running the dictation software), or the Radiologist is not currently dictating or correcting a report, display the “inactive” color (gray), and return to step 1.
4. For each examination associated with the current report, determine which rules apply to that examination. For example, if an MRI of the brain with contrast and MR angiography of the brain are being dictated in the current report, the rule sets for intravenous contrast, and reconstructions of an angiography examination, would apply.
5. Remove any redundant rules. For example, if examinations of the brain and spine without contrast are being dictated in the current report, the non-contrast statement rule need be invoked only once.
6. Determine, from the dictation software, whether a resident or fellow is associated with the current report; if so, add the teaching rule to the set of applicable rules.
7. Check all rules in the set of applicable rules, such as using a natural-language processing algorithm. For each rule, generate text summarizing compliance or noncompliance, as well as a Boolean variable indicating the satisfaction of that rule.
8. Send the results to the user interface.
9. The user interface constructs a tooltip from the rule-compliance statements, and uses similar methods to construct text shown in the main Aulev™ window (e.g., “Needs: teaching statement, contrast type and amount”).
10. The user interface colors the main panel red if any applicable rules are not satisfied; otherwise, it colors the main panel green.
11. Start over at step 1, every 500 msec, or at another suitable interval.

This loop requires approximately between 4 and 100 ms to complete, causing no noticeable lag or change in performance of the workstation while dictating. As reports are modified, either by typing, cutting, pasting, or dictating, Aulev™'s main panel will seemingly immediately change between red and green to indicate compliance, and the text in the main panel will be updated to specify which aspects of the report require correction. Any findings, such as the presence of an abdominal aortic aneurysm, that trigger recommendation rules, cause Aulev™'s main panel to turn blue; if the radiologist clicks on the blue area, a dialog appears allowing insertion of best-practice recommendations for that abnormality.

In one exemplary embodiment, the system and method determines if a field of the current report, such as the sex of a patient is female (i.e. PatientSex is female) by analyzing the extracted metadata. If true, the system automatically removes redundant rules associated with the sex of the patient and other rules of the examination. The system and method further determines which personnel (e.g., resident, fellow) is associated with the report, and checks all rules in the set of applicable rules using a natural-language processing algorithm that are associated with the sex of the patient.

In an additional exemplary embodiment, the system and method determines if a field of the current report is Report-Requirement-3D (i.e. if ReportContains(“3-Dimensional”) is true by analyzing the extracted metadata. In another exemplary embodiment, the system and method determines if a field of the current report is AbdominalAorticAneurysm-Units is “cm” and if field AbdominalAorticAneurysm-Size<2.6. If each are true, then the system and method checks all rules in the set of applicable rules using a natural-language processing algorithm that are associated with AbdominalAorticAneurysm-Units and AbdominalAorticAneurysm-Size. Thus, the system and method would set a Boolean variable AbdominalAorticAneurysm-Recommendation to “No follow-up required.”

Although checking could be performed once at the time a report is signed, this approach allows the Radiologist to see in real time which aspects of the report are noncompliant, which makes correction more efficient.

Currently, Aulev™ obtains its information by examining the windows in the dictation software, and by communicating with a radiology information system (RIS) (e.g., via PowerScribe®'s API) over the network. This tight integration between Aulev™ and PowerScribe® increases performance, and, more important, simplifies distribution to any Radiologist using PowerScribe®, regardless of the PACS or RIS.

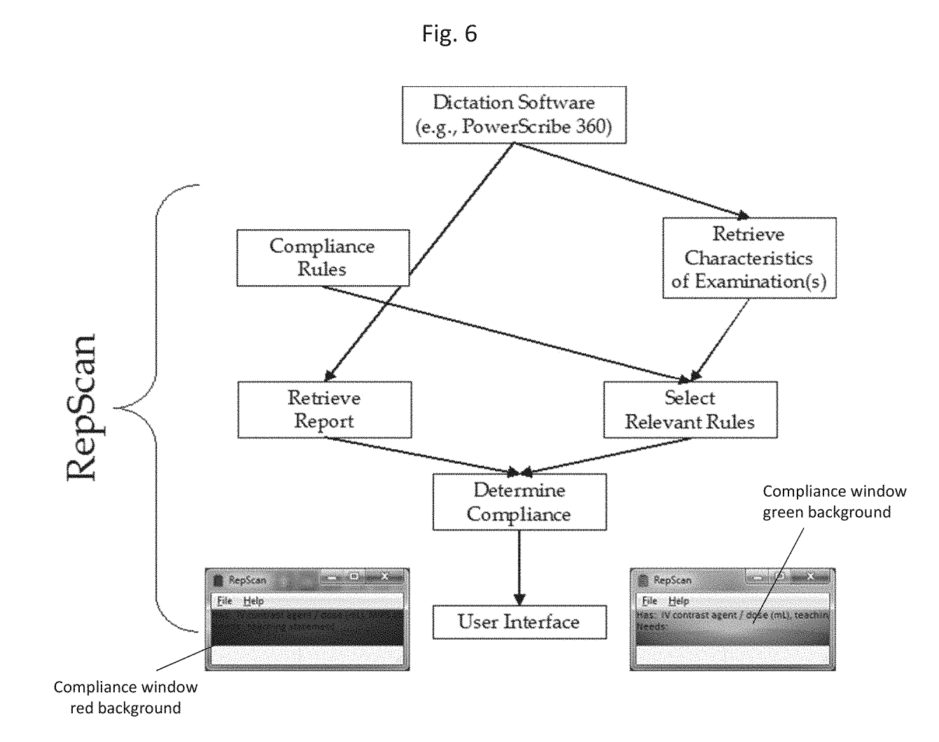

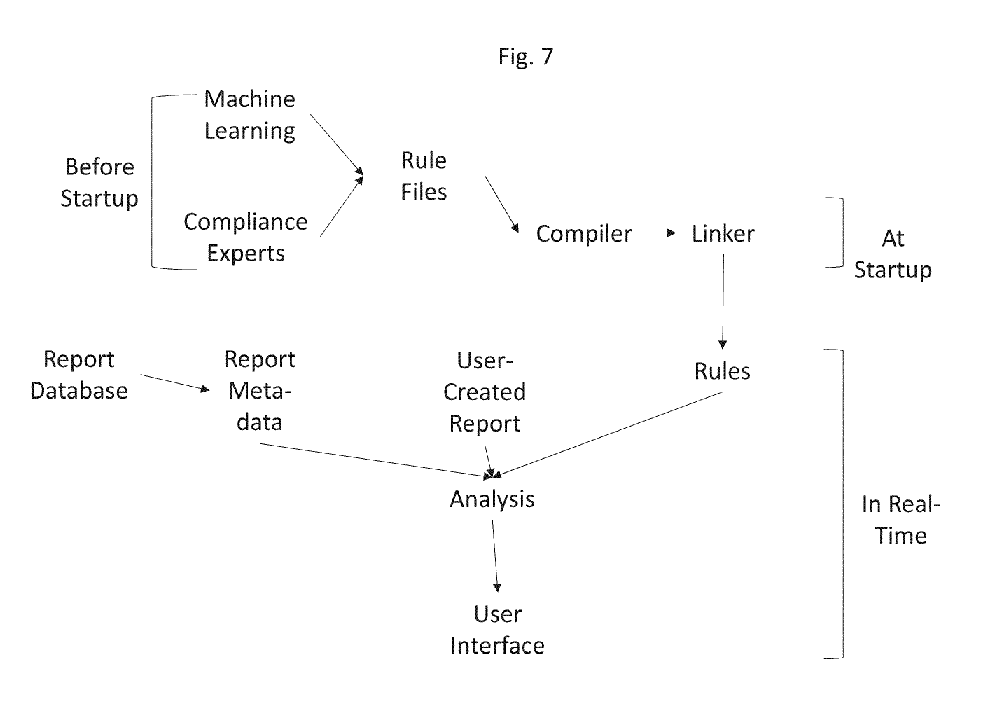

FIGS. 6 and 7 show a schematic diagram of the basic operations of Aulev™ software and corresponding Aulev compliance-windows implemented by the inventor herein.

Additional figures further describe the current embodiment and environment of the present invention, as discussed herein.

The system presented above provides radiologists and other professionals with a tool helping them to create complete, valid reports. The systems and methods disclosed herein are not limited by the type of professional and the type of report. The skilled artisan would understand that these systems may be used by any professional faced with the problem of creating dictated or text reports that must comply with a set of criteria. In addition, the system may be adapted for implementing the recommendation rules to improve the practice of the physician or other professionals. This function may include additional report-type databases, such as those described above, but configured with practice recommendation rules. The system may operate the additional databases sequentially or simultaneously with other report-type databases, as described above.

While the exemplary embodiments have been shown and described, it will be understood by those skilled in the art that various changes in form and details may be made thereto without departing from the spirit and scope of the present disclosure as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

1. A method for real-time validation of a dictated report dictated by a user, the method comprising:
receiving, for each of a plurality of report types, (i) report-type criteria that define the report type and (ii) compliance rules associated with the report type;
receiving report text from a composition system in which the voice of the user is transcribed as the report text in real-time as the user dictates the dictated report;
extracting metadata from the received report text;
determining a report type of the dictated report by comparing the extracted metadata to the report-type criteria received for each of the plurality of report types;
determining compliance with the compliance rules associated with the determined report type by applying the compliance rules to the received report text; and
transmitting, to a user-interface, information indicating the determined compliance with the compliance rules associated with the determined report type.

2. The method according to claim 1, further comprising:
when the determined compliance indicates that the compliance rules are not satisfied, displaying, via the user interface, one or more actions required by the user to satisfy the compliance rules; and
when the determined compliance indicates that the compliance rules are satisfied, displaying, via the user interface, that the dictated report is valid.

3. The method according to claim 1, further comprising periodically repeating the steps of receiving, extracting, determining a report type, determining compliance, and transmitting.

4. The method according to claim 1, wherein for each of the plurality of report types, the report-type criteria and the compliance rules are defined by a human.

5. The method according to claim 1, wherein for each of the plurality of report types, the report-type criteria and the compliance rules are defined by a processor programmed to determine associations between metadata, report text, report types, and report validity.

6. The method according to claim 1, wherein for each of the plurality of report types, the compliance rules include (i) report-validation requirements associated with the report type and (ii) report-validation criteria for the report-validation requirement associated with the report type.

7. The method according to claim 1, wherein for each of the plurality of report types, the compliance rules include (i) recommendation-applicability criteria that determine recommendations applicable with the report type and (ii) recommendation-recommendation criteria for the recommendations applicable with the report type.

8. A system for real-time validation a dictated report dictated by a user, the system comprising:
a composition system in which a voice of the user is transcribed as report text in real-time as the user dictates the dictated report; and
a processor programmed to perform the following steps:
receiving, for each of a plurality of report types, (i) report-type criteria that define the report type and (ii) compliance rules associated with the report type;
receiving report text from a composition system in which the voice of the user is transcribed as the report text in real-time as the user dictates the dictated report;
extracting metadata from the received report text;
determining a report type of the dictated report by comparing the extracted metadata to the report-type criteria received for each of the plurality of report types;
determining compliance with the compliance rules associated with the determined report type by applying the compliance rules to the received report text; and
transmitting, to a user-interface, information indicating the determined compliance with the compliance rules associated with the determined report type.

9. The system according to claim 8, further comprising a user-interface including a display, the user interface being configured to receive the transmitted information indicating the determined compliance,
wherein when the determined compliance indicates that the compliance rules are not satisfied, the display displays one or more actions required by the user to satisfy the compliance rules; and
wherein when the determined compliance indicates that the compliance rules are satisfied, the display displays that the dictated report is valid.

10. The system according to claim 8, wherein the processor periodically repeats the steps of receiving, extracting, determining a report type, determining compliance, and transmitting.

11. The system according to claim 8, wherein for each of the plurality of report types, the report-type criteria and the compliance rules are defined by a human.

12. The system according to claim 8, wherein for each of the plurality of report types, the report-type criteria and the compliance rules are defined by a processor programmed to determine associations between metadata, report text, report types, and report validity.

13. The system according to claim 8, wherein for each of the plurality of report types, the compliance rules include (i) report-validation requirements associated with the report type and (ii) report-validation criteria for the report-validation requirement associated with the report type.

14. The system according to claim 8, wherein for each of the plurality of report types, the compliance rules include (i) recommendation-applicability criteria that determine recommendations applicable with the report type and (ii) recommendation-recommendation criteria for the recommendations applicable with the report type.
